(19) United States
(12) Patent Application Publication
(10) Pub. No.: US 2016/0051167 A1
(43) Pub. Date: Feb. 25, 2016

(54) SYSTEM AND METHOD FOR ACTIVITY CLASSIFICATION

(71) Applicant: InvenSense, Inc., San Jose, CA (US)

(72) Inventors: Sankalita Saha, Redwood Shores, CA (US); Hemabh Shekar, San Jose, CA (US); Shang Hung Lin, San Jose, CA (US); Chih-Chieh Geoff Chang, Mountain View, CA (US)

(21) Appl. No.: 14/464,999

(22) Filed: Aug. 21, 2014

(51) Int. Cl. A61B 5/11

(52) U.S. Cl. CPC A61B 5/1123

(57) ABSTRACT

A system and method for efficiently and accurately classifying user activity. In a non-limiting example, accelerometer signals and/or gyroscope sensor signals may be analyzed to classify user activity, for example, that of a user of a handheld and/or wearable device. Information from additional sources and sensors (e.g., other inertial sensors, non-inertial sensors, passive sensors, etc.) may also be utilized.

### CROSS-REFERENCE TO RELATED APPLICATIONS/INCORPORATION BY REFERENCE

This patent application is related to patent application Ser. No. 13/648,963 filed Oct. 10, 2012, and titled “ACTIVITY CLASSIFICATION IN A MULTI-AXIS ACTIVITY MONITOR DEVICE,” the entire contents of which are hereby incorporated herein by reference in their entirety.

### FEDERALLY SPONSORED RESEARCH OR DEVELOPMENT

[Not Applicable]

### SEQUENCE LISTING

[Not Applicable]

### MICROFICHE/COPYRIGHT REFERENCE

[Not Applicable]

### BACKGROUND

Knowledge of user activity is often important. Such knowledge generally, however, comes at a high cost, for example a high cost with respect to processor cycles, energy, memory code space, semiconductor real estate, etc. Further limitations and disadvantages of conventional and traditional approaches will become apparent to one of skill in the art, through comparison of such approaches with the disclosure as set forth in the remainder of this application with reference to the drawings.

### BRIEF DESCRIPTION OF SEVERAL VIEWS OF THE DRAWINGS

FIG. 1 shows a block diagram of an example electronic device comprising activity classification capability, in accordance with various aspects of the present disclosure.

FIG. 2 shows an example activity classification system, in accordance with various aspects of the present disclosure.

FIG. 3 shows an example multiclass decision tree, in accordance with various aspects of the present disclosure.

FIG. 4 shows an example multiclass decision tree, in accordance with various aspects of the present disclosure.

FIG. 5 shows an example activity classification system, in accordance with various aspects of the present disclosure.

FIG. 6 shows an example multiclass decision tree, in accordance with various aspects of the present disclosure.

### SUMMARY

Various aspects of this disclosure comprise methods and systems for efficiently and accurately classifying user activity. In a non-limiting example, accelerometer signals and/or gyroscope sensor signals may be analyzed to classify user activity, for example, that of a user of a handheld and/or wearable device. Information from additional sources and sensors (e.g., other inertial sensors, non-inertial sensors, passive sensors, etc.) may also be utilized.

### DETAILED DESCRIPTION OF VARIOUS ASPECTS OF THE DISCLOSURE

The following discussion presents various aspects of the present disclosure by providing various examples thereof. Such examples are non-limiting, and thus the scope of various aspects of the present disclosure should not necessarily be limited by any particular characteristics of the provided examples. In the following discussion, the phrases “for example” and “e.g.” and “exemplary” are non-limiting and are generally synonymous with “by way of example and not limitation,” “for example and not limitation,” and the like.

The following discussion may at times utilize the phrase “A and/or B.” Such phrase should be understood to mean just A, or just B, or both A and B. Similarly, the phrase “A, B, and/or C” should be understood to mean just A, just B, just C, A and B, A and C, B and C, or all of A and B and C.

The following discussion may at times utilize the phrases “operable to,” “operates to,” and the like in discussing functionality performed by particular hardware, including hardware operating in accordance with software instructions. The phrases “operates to,” “is operable to,” and the like include “operates when enabled to.” For example, a module that operates to perform a particular operation, but only after receiving a signal to enable such operation, is included by the phrases “operates to,” “is operable to,” and the like.

The following discussion may at times refer to various system or device functional modules. It should be understood that the functional modules were selected for illustrative clarity and not necessarily for providing distinctly separate hardware and/or software modules. For example, any one or more of the modules discussed herein may be implemented by shared hardware, including for example a shared processor. Also for example, any one or more of the modules discussed herein may share software portions, including for example subroutines. Additionally for example, any one or more of the modules discussed herein may be implemented with independent dedicated hardware and/or software. Accordingly, the scope of various aspects of this disclosure should not be limited by arbitrary boundaries between modules unless explicitly claimed. Additionally, it should be understood that when the discussion herein refers to a module performing a function, the discussion is generally referring to either a pure hardware module implementation and/or a processor operating in accordance with software. Such software may, for example, be stored on a non-transitory machine-readable medium.

In various example embodiments discussed herein, a chip is defined to include at least one substrate typically formed from a semiconductor material. A single chip may for example be formed from multiple substrates, where the substrates are mechanically bonded to preserve the functionality. Multiple chip (or multi-chip) includes at least 2 substrates, wherein the 2 substrates are electrically connected, but do not require mechanical bonding.

A package provides electrical connection between the bond pads on the chip (or for example a multi-chip module) to a metal lead that can be soldered to a printed circuit board (or PCB). A package typically comprises a substrate and a cover. An Integrated Circuit (IC) substrate may refer to a silicon substrate with electrical circuits, typically CMOS circuits. A MEMS substrate provides mechanical support for the MEMS structure(s). The MEMS structural layer is attached to the MEMS substrate. The MEMS substrate is also referred to as handle substrate or handle wafer. In some embodiments, the handle substrate serves as a cap to the MEMS structure.

In the described embodiments, an electronic device incorporating a sensor may, for example, employ a motion tracking module also referred to as Motion Processing Unit (MPU) that includes at least one sensor in addition to electronic circuits. The at least one sensor may comprise any of a variety of sensors, such as for example a gyroscope, a compass, a magnetometer, an accelerometer, a microphone, a pressure sensor, a proximity sensor, a moisture sensor, a temperature sensor, a biometric sensor, or an ambient light sensor, among others known in the art.

Some embodiments may, for example, comprise an accelerometer, gyroscope, and magnetometer or other compass technology, which each provide a measurement along three axes that are orthogonal relative to each other, and may be referred to as a 9-axis device. Other embodiments may, for example, comprise an accelerometer, gyroscope, compass, and pressure sensor, and may be referred to as a 10-axis device. Other embodiments may not include all the sensors or may provide measurements along one or more axes.

The sensors may, for example, be formed on a first substrate. Various embodiments may, for example, include solid-state sensors and/or any other type of sensors. The electronic circuits in the MPU may, for example, receive measurement outputs from the one or more sensors. In various embodiments, the electronic circuits process the sensor data. The electronic circuits may, for example, be implemented on a second silicon substrate. In some embodiments, the first substrate may be vertically stacked, attached and electrically connected to the second substrate in a single semiconductor chip, while in other embodiments, the first substrate may be disposed laterally and electrically connected to the second substrate in a single semiconductor package.

In an example embodiment, the first substrate is attached to the second substrate through wafer bonding, as described in commonly owned U.S. Pat. No. 7,104,129, which is hereby incorporated herein by reference in its entirety, to simultaneously provide electrical connections and hermetically seal the MEMS devices. This fabrication technique advantageously enables technology that allows for the design and manufacture of high performance, multi-axis, inertial sensors in a very small and economical package. Integration at the wafer-level minimizes parasitic capacitances, allowing for improved signal-to-noise relative to a discrete solution. Such integration at the wafer-level also enables the incorporation of a rich feature set which minimizes the need for external amplification.

In the described embodiments, raw data refers to measurement outputs from the sensors which are not yet processed. Motion data refers to processed raw data. Processing may, for example, comprise applying a sensor fusion algorithm or applying any other algorithm. In the case of a sensor fusion algorithm, data from one or more sensors may be combined and/or processed to provide an orientation of the device. In the described embodiments, a MPU may include processors, memory, control logic and sensors among structures.

Activity classification, for example for an electronic device, is useful. For example, an operating system of a device may operate the device in different respective manners when the user of the device is engaged in different activities. For example, when a user is riding in a car, particular features of a mobile phone may be disabled and/or operated in a different mode. Also for example, when a user is walking or running, particular features of a mobile phone, for example pedometer features, may be turned on and/or implemented at a higher level.

Accordingly, various aspects of the present disclosure provide non-limiting examples of methods and systems for efficiently classifying user activity. The discussion will now turn to discussing the attached figures.

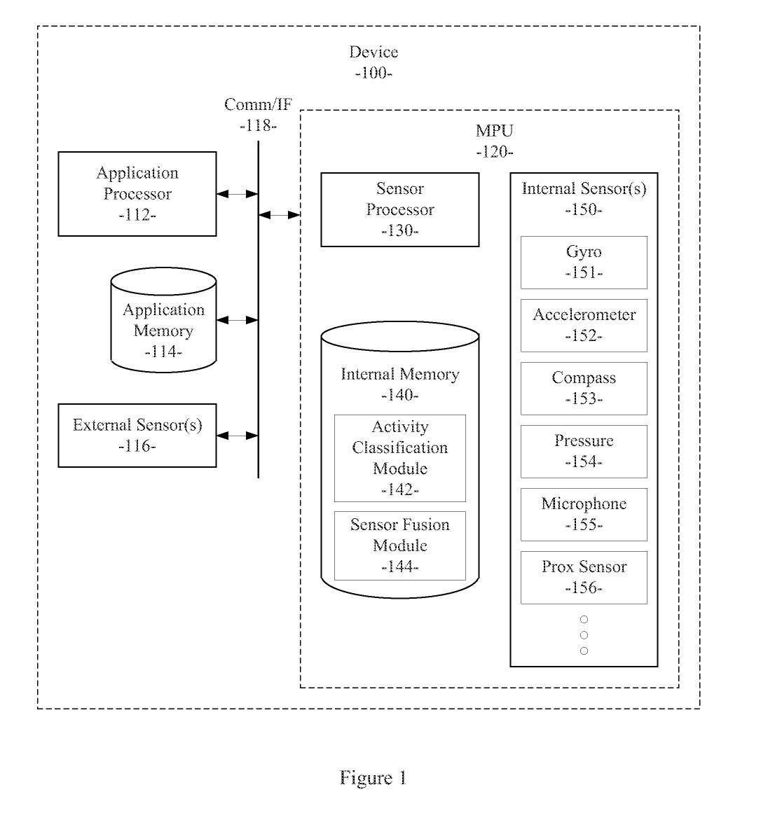

Turning first to FIG. 1, such figure shows a block diagram of an example electronic device 100 comprising activity classification capability, in accordance with various aspects of the present disclosure. As will be appreciated, the device 100 may be implemented as a device or apparatus, such as a handheld and/or wearable device that can be moved in space by a user and its motion and/or orientation in space therefore sensed. For example, such a handheld device may be a mobile phone (e.g., a cellular phone, a phone running on a local network, or any other telephone handset), wired telephone (e.g., a phone attached by a wire and/or optical tether), personal digital assistant (PDA), pedometer, personal activity and/or health monitoring device, video game player, video game controller, navigation device, mobile internet device (MID), personal navigation device (PND), digital still camera, digital video camera, binoculars, telephoto lens, portable music, video, or media player, remote control, or other handheld device, or a combination of one or more of these devices.

In some embodiments, the device 100 may be a self-contained device that comprises its own display and/or other output devices in addition to input devices as described below. However, in other embodiments, the device 100 may function in conjunction with another portable device or a non-portable device such as a desktop computer, electronic tabletop device, server computer, etc., which can communicate with the device 100, e.g., via network connections. The device 100 may, for example, be capable of communicating via a wired connection using any type of wire-based communication protocol (e.g., serial transmissions, parallel transmissions, packet-based data communications), wireless connection (e.g., electromagnetic radiation, infrared radiation or other wireless technology), or a combination of one or more wired connections and one or more wireless connections.

As shown, the example device 100 comprises an MPU 120, application (or host) processor 112, application (or host) memory 114, and may comprise one or more sensors, such as external sensor(s) 116. The application processor 112 may, for example, be configured to perform the various computations and operations involved with the general function of the device 100 (e.g., running applications, performing operating system functions, performing power management functionality, controlling user interface functionality for the device 100, etc.). The application processor 112 may, for example, be coupled to MPU 120 through a communication interface 118, which may be any suitable bus or interface, such as a peripheral component interconnect express (PCIe) bus, a universal serial bus (USB), a universal asynchronous receiver/transmitter (UART) serial bus, a suitable advanced microcontroller bus architecture (AMBA) interface, an Inter-Integrated Circuit (I2C) bus, a serial digital input output (SDIO) bus, or other equivalent. The application memory 114 may comprise programs, drivers or other data that utilize information provided by the MPU 120. Details regarding example suitable configurations of the application (or host) processor 112 and MPU 120 may be found in co-pending, commonly owned U.S. patent application Ser. No. 12/106,921, filed Apr. 21, 2008, which is hereby incorporated by reference in its entirety.

In this example embodiment, the MPU 120 is shown to comprise a sensor processor 130, internal memory 140 and one or more internal sensors 150. The internal sensors 150 may, for example, comprise a gyroscope 151, an accelerometer 152, a compass 153 (for example a magnetometer), a pressure sensor 154, a microphone 155, a proximity sensor 156, etc.). Though not shown, the internal sensors 150 may comprise any of a variety of sensors, for example, a temperature sensor, light sensor, moisture sensor, biometric sensor, etc. The internal sensors 150 may, for example, be implemented as MEMS-based motion sensors, including inertial sensors such as a gyroscope or accelerometer, or an electromagnetic sensor such as a Hall effect or Lorentz field magnetometer. At least a portion of the internal sensors 150 may also, for example, be based on sensor technology other than MEMS technology (e.g., CMOS technology, etc.). As desired, one or more of the internal sensors 150 may be configured to provide raw data output measured along three orthogonal axes or any equivalent structure. The internal memory 114 may store algorithms, routines or other instructions for processing data output by one or more of the internal sensors 150, including the activity classification module 142 and sensor fusion module 144, as described in more detail herein. If provided, external sensor(s) 116 may comprise one or more sensors, such as accelerometers, gyroscopes, magnetometers, pressure sensors, microphones, proximity sensors, and ambient light sensors, biometric sensors, temperature sensors, and moisture sensors, among other sensors. As used herein, an internal sensor generally refers to a sensor implemented, for example using MEMS techniques, for integration with the MPU 120 into a single chip. Also, an external sensor as used herein generally refers to a sensor carried on-board the device 100 that is not integrated into the MPU 120.

Even though various embodiments may be described herein in the context of internal sensors implemented in the MPU 120, these techniques may be applied to a non-integrated sensor, such as an external sensor 116, and likewise the activity classification module 142 and/or sensor fusion module 144 may be implemented using instructions stored in any available memory resource, such as for example the application memory 114, and may be executed using any available processor, such as the application processor 112. Still further, the functionality performed by the activity classification module 142 may be implemented using any combination of hardware, firmware and software.

As will be appreciated, the application (or host) processor 112 and/or sensor processor 130 may be one or more microprocessors, central processing units (CPUs), microcontrollers or other processors which run software programs for the device 100 and/or for other applications related to the functionality of the device 100. For example, different software application programs such as menu navigation software, games, camera function control, navigation software, and phone or a wide variety of other software and functional interfaces can be provided. In some embodiments, multiple different applications can be provided on a single device 100, and in some of those embodiments, multiple applications can run simultaneously on the device 100. Multiple layers of software can, for example, be provided on a computer readable medium such as electronic memory or other storage medium such as hard disk, optical disk, flash drive, etc., for use with application processor 112 and sensor processor 130. For example, an operating system layer can be provided for the device 100 to control and manage system resources in real time, enable functions of application software and other layers, and interface application programs with other software and functions of the device 100. In various example embodiments, one or more motion algorithm layers may provide motion algorithms for lower-level processing of raw sensor data provided from internal or external sensors. Further, a sensor device driver layer may provide a software interface to the hardware sensors of the device 100. Some or all of these layers can be provided in the application memory 114 for access by the application processor 112, in internal memory 140 for access by the sensor processor 130, or in any other suitable architecture (e.g., including distributed architectures).

In some example embodiments, it will be recognized that the example architecture depicted in FIG. 1 may provide for activity classification to be performed using the MPU 120 and might not require involvement of the application processor 112 and/or application memory 114. Such example embodiments may, for example, be implemented with one or more internal sensor sensors 150 on a single substrate. Moreover, as will be described below, the activity classification techniques may be implemented using computationally efficient algorithms to reduce processing overhead and power consumption.

As discussed herein, various aspects of this disclosure may, for example, comprise processing various sensor signals indicative of device orientation. Non-limiting examples of such signals are signals that indicate accelerometer orientation along the z-axis (or gravitational axis) in a world coordinate system.

In an example implementation, an accelerometer and/or associated circuitry may output a vector indicative of device (or accelerometer) orientation. Such a vector may, for example, initially be expressed in a body (or device) coordinate system. Such a vector may be processed by a transformation function, for example based on sensor fusion calculations, that transforms the accelerometer vector to a world coordinate system. For example, an accelerometer vector A_{b}=[A_{b}^{x}, A_{b}^{y}, A_{b}^{z}] in body (or device) coordinates may be transformed to an accelerometer vector A_{w}=[A_{w}^{x}, A_{w}^{y}, A_{w}^{z}] in world coordinates.

Portions of the following discussion may generally focus on the z-axis component of the accelerometer vector in the world coordinate system, for example A_{w}^{z}. It should be noted, however, that the scope of this disclosure is not limited by the particular signal(s) and/or coordinate system(s) discussed herein.

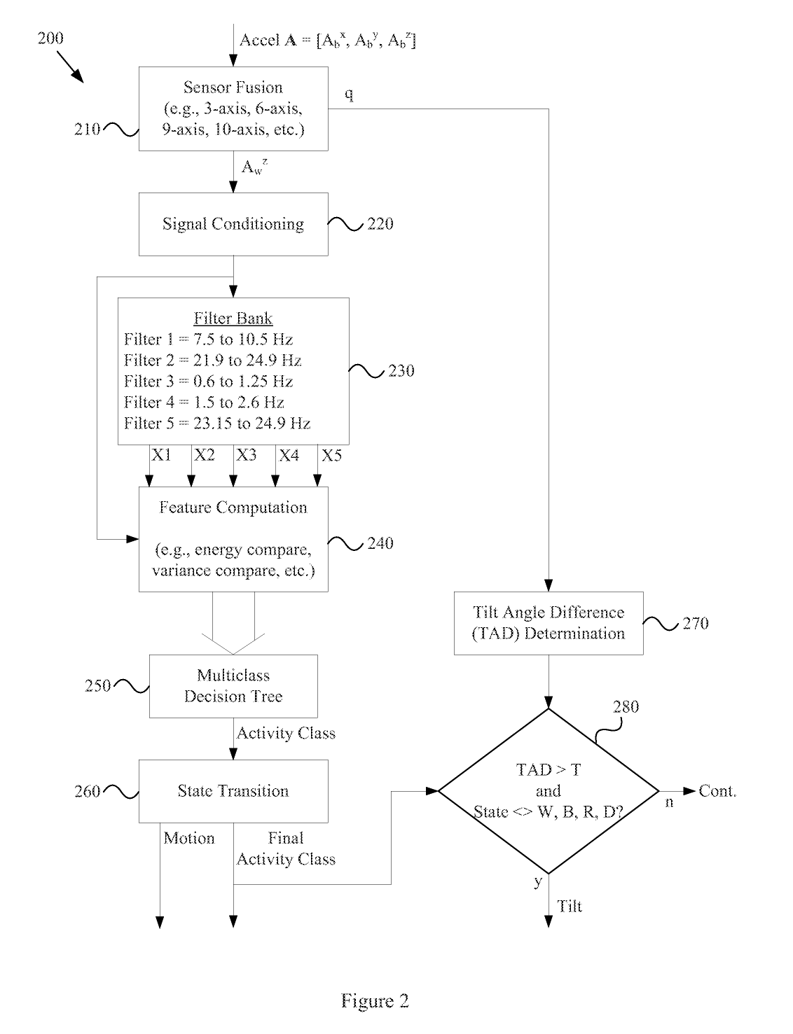

As mentioned herein, the activity classification module 142 may be implemented by a processor (e.g., the sensor processor 130) operating in accordance with software instructions (e.g., the activity classification module software 142 stored in the internal memory 140), or by a pure hardware solution. The discussion of FIGS. 2-6 will provide further example details of at least the operation of the sensor fusion module 144 and the activity classification module 142. It should be understood that any or all of the functional modules discussed herein may be implemented in a pure hardware implementation and/or by a processor operating in accordance with software instructions. It should also be understood that any or all software instructions may be stored in a non-transitory computer-readable medium.

Various aspects of this disclosure comprise processing various frequency bands of one or more signals indicative of device orientation and/or movement. Examples of such signals may, for example, comprise a signal that indicates accelerometer orientation along the z-axis (or gravitational axis) in a world coordinate system, signals that indicate accelerometer orientation along any or all axes in a world coordinate system, gyroscope signals along any or all axes in a world coordinate system, etc.

One of the challenges in accurately identifying a user activity with a handheld device (e.g., a mobile phone), and even a wearable device (e.g., a watch or belt-mounted device), is that the orientation of the device or how the user carries the device varies from user to user and from moment to moment. For example, a user may carry a device in-hand, in a pocket, on a belt, on a wrist, etc.

Through empirical studies, it has been determined that the A_{w}^{z} signal includes a variety of frequency spectrum components that are associated with particular user activities. For example, the A_{w}^{z} signal will generally comprise one or more frequency components (e.g., the existence or absence thereof) associated with walking and/or running, one or more frequency components (e.g., the existence or absence thereof) associated with biking, one or more frequency components (e.g., the existence or absence thereof) associated with driving and/or riding in a motor vehicle, one or more frequency components (e.g., the existence or absence thereof) associated with being generally stationary, etc. The same is true with other accelerometer signals, with gyroscope signals, with proximity sensor signals, with microphone signals, with pressure sensor signals, and other sensor signals. Thus, although for illustrative purposes, various parts of the following discussion will focus on the A_{w}^{z} signal, the scope of this disclosure is not limited to the analysis of such signal.

Depending on the manner in which a user is carrying and/or using a device, different frequency components may dominate. Thus, the following discussion presents many examples of systems that may each be applied to handheld and/or wearable devices. The respective systems 100, 200, and 500 shown in FIGS. 1, 3, and 5, and discussed herein, may share any or all characteristics with each other.

Turning next to FIG. 2, such figure shows an example activity classification system 200 in accordance with various aspects of the present disclosure. The example system 200 may, for example, be used to classify an activity of a user using a handheld device (e.g., a mobile telephone, PDA, camera, portable media player, gaming device, etc.). Note, however, that the system 200 is not limited to handheld devices, for example being readily applicable to wearable devices (e.g., a watch, a headband, an armband, a belt-mounted device, eyeglasses, etc.) and other devices. The example system 200 may, for example, share any or all characteristics with the example device 100 illustrated in FIG. 1 and discussed above. For example, the system 200 or any portion thereof may, for example, be implemented with the sensor processor 130 of FIG. 1 operating in accordance with software instructions in the sensor fusion software module 144 and activity classification software module 142 stored in the internal memory 140. Also for example, the system 200 or any portion thereof may be implemented with the application (or host) processor 112 operating in accordance with software instructions stored in the application memory 114.

As discussed above, an accelerometer signal (e.g., a vector) may be expressed in body coordinates. Such an accelerometer signal may be input to the sensor fusion module 210, which transforms the accelerometer signal into the world (or inertial) coordinate system, for example based on a rotation matrix that the sensor fusion module 210 determines in real-time based on one or more of accelerometer signals, gyroscope signals, compass signals, etc. In this particular example system, the primary focus will be on the z-axis component of the accelerometer vector in the world coordinate system, A_{w}^{z}, which is shown as an output from the sensor fusion module 210 in FIG. 2.

The signal A_{w}^{z} may, for example, be viewed as a discrete time signal that is updated at a sensor update rate of the accelerometer signal. The calculations and determinations herein may also be performed at the sensor rate. Note, however, that any of a variety of sensor signal update and/or activity class determination rates may be utilized. For example, during various contexts, for example when it is known that a user is engaged in a particular activity (e.g., for a substantial period of time), the sensor update rate and/or class determination rate may in general be slowed to conserve energy. Conversely, when nothing is known about the user activity, for example when a device is reset or powered on, or when a sudden change in the device usage is detected, or when an application is initiated that needs to know the user's activity, a relatively higher signal update rate and/or activity class determination rate may be utilized.

From the sensor fusion module 210, the signal A_{w}^{z} may, for example, be provided to a signal conditioning module 220 to prepare the A_{w}^{z} signal for subsequent processing. The signal conditioning module 220 may, for example, remove noise from the A_{w}^{z} signal, restore shape to a clipped A_{w}^{z} signal, adjust the amplitude of the A_{w}^{z} signal (e.g., removing a known bias, scaling the amplitude of the signal for subsequent processing, etc.), etc.

The conditioned A_{w}^{z} signal may then be provided to a filter bank module 230. As mentioned herein, the spectral content of the A_{w}^{z} signal will change based on user activity. The filter bank module 230 may, for example, comprise a plurality of filters that operate to isolate frequency ranges of interest in the A_{w}^{z} signal. The example filter bank module 230 is illustrated with five filters, but may comprise any number of filters. The example filters are bandpass filters with the following corner frequencies:
Filter 1 (X1): 7.5 to 10.5 Hz
Filter 2 (X2): 21.9 to 24.9 Hz
Filter 3 (X3): 0.6 to 1.25 Hz
Filter 4 (X4): 1.5 to 2.6 Hz
Filter 5 (X5): 23.15 to 24.9 Hz

The filter characteristics are merely examples. The example filter characteristics were found empirically to work well, but the scope of the disclosure is not limited to the number of filters nor by the respective characteristics of each of such filters. The number of filters and/or characteristics of the filters may change depending on device type, depending on the user, depending on real-time activity indicated by sensor signals or other device signals, etc. Other non-limiting filter bank examples are provided herein.

The filtered signal outputs from the filter bank 230 (e.g., Filter 1 to Filter 5), labeled X1-X5, are provided to a feature computation module 240. The feature computation module 240 may, for example, process the inputs X1-X5 (e.g., individually and/or in any combination) to identify features of interest in the filtered signals. The feature computation module 240 may, for example, determine indications of the respective energy found in each of the input signals X1-X5 (e.g., signal amplitudes, squared signal amplitudes, signal amplitudes integrated over time, etc.). The feature computation module 240 may determine and/or estimate the signal energy in any of a variety of manners. For example, the feature computation module 240 may estimate signal energy based on amplitude of a signal. For example, a running scaled sum of signal amplitude may be utilized:

z₂=abs(Xn)

e1ₙ=e1ₙ₋₁+α(z₂−e1ₙ₋₁)

Also for example, a running scaled sum of the square of a signal may be used. For example:

z₃=(Xn)²

e2ₙ=e2ₙ₋₁+α(z₃−e2ₙ₋₁)

The above equations are merely examples, and not meant to be limiting. For example, an IIR filter may be used to smooth out the energy estimations. Alternatively, such an IIR filter might not be used, or a different type of filter (e.g., an FIR filter) may be used. For example, a moving average may also be used.

The feature computation module 240 may also combine features, for example features of individual filtered signals, to calculate more complex features. For example, the feature computation module 240 may calculate a feature as a sum and/or ratio of other features. For example, a set of features may comprise:
Feature 1 (F1): Filter 1 energy
Feature 2 (F2): Filter 2 energy
Feature 3 (F3): Feature 1/Feature 2
Feature 4 (F4): Filter 3 energy+Filter 4 energy
Feature 5 (F5): Filter 5 energy
Feature 6 (F6): Feature 4/Feature 5

Additionally for example, in various implementations, the feature computation module 240 may determine (or compute) one or more indications of variability (e.g., of the filtered signals, of combinations of the filtered signals, etc.). For example, the feature computation module 240 may determine the variance (or standard deviation) of the A_{w}^{z} signal (e.g., after signal conditioning as shown in FIG. 2, or before signal conditioning). For example, continuing the example above:
Feature 0 (F0): Variance of A_{w}^{z}

The feature computation module 240 may output the determined features (e.g., metrics or characteristics), which may then for example be provided to a multiclass decision tree module 250. The multiclass decision tree module 250 may, for example, analyze the features received from the feature computation module 240 to identify an activity class.

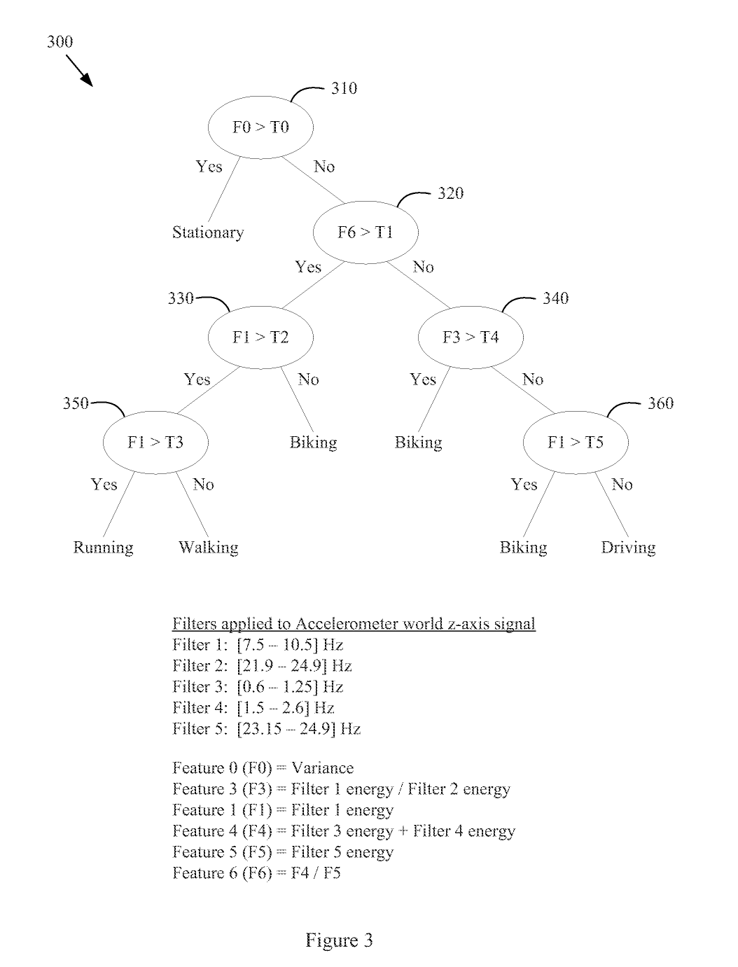

The leaf nodes of the decision tree, for example, may represent an activity class. An interior node of the decision tree may, for example, represent a plurality of activity classes and/or a decision point in the tree traversal. For example, an interior node may correspond to both running and walking. In such a scenario, the interior node may be reached while traversing the decision tree because one or more features indicate a threshold amount of detected energy (e.g., in filtered signals X1 and/or X2) in one or more frequency ranges associated with running and walking activity. Other features (e.g., signal magnitude, harmonic frequency content, etc.) may be then analyzed, and/or same features compared to different threshold, to further refine the determined activity class between running and walking Note that there may be a plurality of decision trees that may be selectively utilized based on various conditions. The output of the multiclass decision tree module 250 may, for example, comprise an identified activity class. An example of a decision tree 300, which may for example be implemented by the multiclass decision tree module 250, is presented with respect to FIG. 3.

Turning now to FIG. 3, such figure shows an example multiclass decision tree 300, in accordance with various aspects of the present disclosure. The multiclass decision tree 300 is merely an example selected for illustrative clarity. Accordingly, the scope of various aspects of this disclosure should not be limited by any characteristics of the multiclass decision tree 300. The multiclass decision tree 300 may, for example be utilized in an implementation of a handheld device. The multiclass decision tree 300 may also, however, be utilized in an implementation of a wearable device or other type of device.

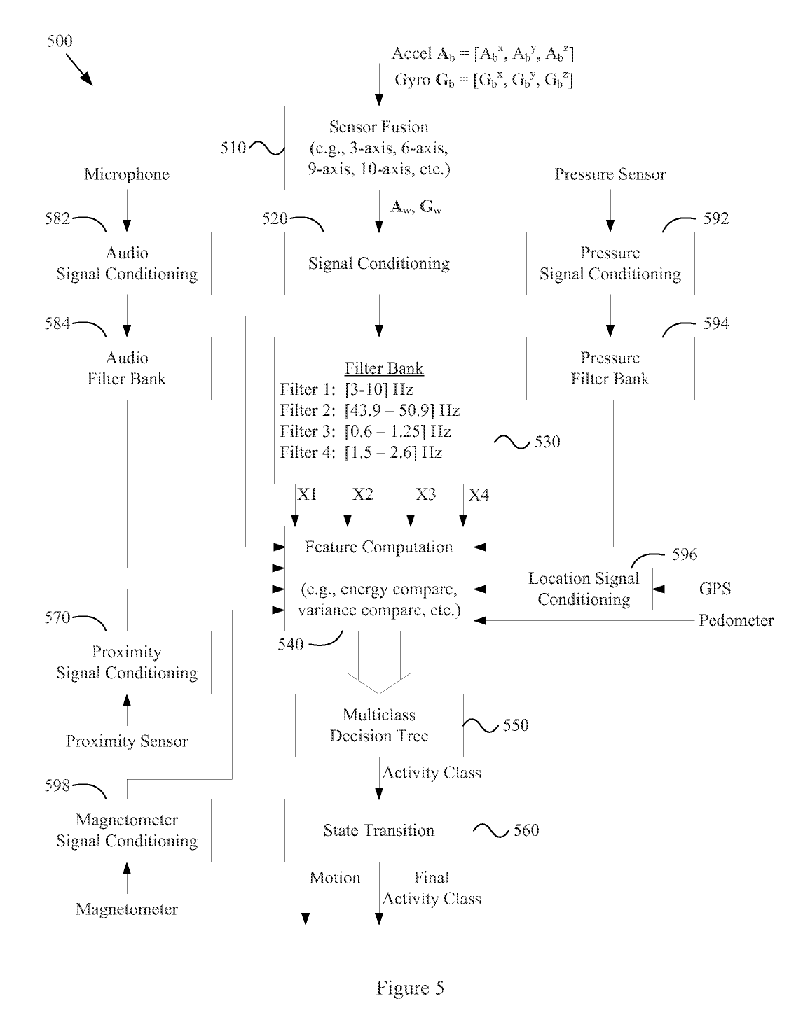

Traversal of the decision tree 300 begins at the root node 310, at which Feature 0 (or F0) is compared to a threshold value T0. In this example, Feature 0 may comprise an indication of variance of the A_{w}^{z} signal. If, for example, Feature 0 exceeds the threshold T0, then traversal of the decision tree 300 proceeds to a leaf node that indicates that the device (e.g., the device 100 of FIG. 1, a device implementing the system 200 of FIG. 2, a device implementing the system 500 of FIG. 5, etc.) is in a stationary state. If, however, Feature 0 does not exceed the threshold T0, then traversal of the decision tree 300 proceeds to a second interior node 320, at which the activity class could be any of walking, running, biking, or driving.

At the second interior node 320, the multiclass decision tree 300 compares Feature 6 (or F6) to a first threshold value T1. In this example, Feature 6 may comprise a ratio of Feature 4 to Feature 5, where Feature 4 may for example comprise a sum of the indications of energy in the Filter 3 signal (e.g., signal X3 from the filter bank 230) and the Filter 4 signal (e.g., signal X4 from the filter bank 230), and Feature 5 may comprise an indication of energy in the Filter 5 signal. If, for example, Feature 6 exceeds the first threshold T1, then traversal of the decision tree 300 proceeds to a third interior node 330, at which the activity class could be any of walking, running, or biking. If, however, Feature 6 does not exceed the first threshold T1, then traversal of the decision tree 300 proceeds to a fourth interior node 340, at which the activity class could be either biking or driving.

At the third interior node 330, the multiclass decision tree 300 compares Feature 1 (or F1) to a second threshold value T2. In this example, Feature 1 may comprise an indication of energy in the Filter 1 signal (e.g., signal X1 from the filter bank 230). If, for example, Feature 1 exceeds the second threshold T2, then traversal of the decision tree 300 proceeds to a fifth interior node 340, at which the activity class could be either running or walking. If, however, Feature 1 does not exceed the second threshold T2, then traversal of the decision tree 300 proceeds to a leaf node that indicates that the device (e.g., the device 100 of FIG. 1, a device implementing the system 200 of FIG. 2, a device implementing the system 500 of FIG. 5, etc.) is associated with a biking state, for example a user of the device is biking with the device.

At the fifth interior node 350, the multiclass decision tree 300 compares Feature 1 (or F1) to a third threshold value T3 (e.g., different from threshold value T2). In this example, F1 may comprise an indication of energy in the Filter 1 signal (e.g., signal X1 from the filter bank 230). If, for example, Feature 1 exceeds the third threshold T3, then traversal of the decision tree 300 proceeds to a leaf node that indicates that the device (e.g., the device 100 of FIG. 1, a device implementing the system 200 of FIG. 2, a device implementing the system 500 of FIG. 5, etc.) is associated with a running state, for example a user of the device is running with the device. If, however, Feature 1 does not exceed the third threshold T3, then traversal of the decision tree 300 proceeds to a leaf node that indicates that the device (e.g., the device 100 of FIG. 1, a device implementing the system 200 of FIG. 2, a device implementing the system 500 of FIG. 5, etc.) is associated with a walking state, for example a user of the device is walking with the device.

At the fourth interior node 340, the multiclass decision tree 300 compares Feature 3 (or F3) to a fourth threshold value T4. In this example, Feature 3 may comprise a ratio of an indication of energy in the Filter 1 signal (e.g., signal X1 from the filter bank 230) to an indication of energy in the Filter 2 signal (e.g., signal X2 from the filter bank 230). If, for example, Feature 3 exceeds the fourth threshold T4, then traversal of the decision tree 300 proceeds to a leaf node that indicates that the device (e.g., the device 100 of FIG. 1, a device implementing the system 200 of FIG. 2, a device implementing the system 500 of FIG. 5, etc.) is associated with a biking state. If, however, Feature 3 does not exceed the fourth threshold T4, then traversal of the decision tree 300 proceeds to a sixth interior node 360, at which the activity class could be either biking or driving.

At the sixth interior node 360, the multiclass decision tree 300 compares Feature 1 (or F1) to a fifth threshold value T5 (e.g., different from the fourth threshold value T4). In this example, F1 may comprise an indication of energy in the Filter 1 signal (e.g., signal X1 from the filter bank 230). If, for example, Feature 1 exceeds the fifth threshold T5, then traversal of the decision tree 300 proceeds to a leaf node that indicates that the device (e.g., the device 100 of FIG. 1, a device implementing the system 200 of FIG. 2, a device implementing the system 500 of FIG. 5, etc.) is associated with a biking state. If, however, Feature 1 does not exceed the fifth threshold T5, then traversal of the decision tree 300 proceeds to a leaf node that indicates that the device (e.g., the device 100 of FIG. 1, a device implementing the system 200 of FIG. 2, a device implementing the system 500 of FIG. 5, etc.) is associated with a driving state, for example a user of the device is driving or riding in a vehicle.

The features and/or thresholds utilized in the decision tree 300 may, for example, be adaptable. For example, as differences between features and thresholds become known (e.g., empirically), the features and/or thresholds may be adjusted. For example, referring to the decision tree 300 traversal through the third node 330, as operation of the device and the decision tree 300 is monitored over time, a better understanding of the differences between biking and (running or walking) for the device or user may result in a shifting of the second threshold T2. Also for example, a better understanding of the differences between running and walking may result in a shifting of the third threshold T3. Additionally, user feedback may be utilized to adjust the parameters of the decision tree 300. For example, information of the selected state may be provided by the device 100 to the user, who may then provide feedback when an incorrect and/or correct state has been selected. The multiclass decision tree module 250 (or other module of the system 200) may then identify the node at which the incorrect decision was made and adjust the feature and/or threshold parameters at such node to reduce the chance that the same error will occur again. In such a manner, operation of the decision tree 300 may be tailored to a device and/or user thereof

The activity class decisions may be performed at any of a variety of rates. The decision rate may, for example, correspond to the sensor update rate, a step rate, regular timed intervals, etc. The decision may, for example, be performed continually, but sampled at another rate. For example, the activity class may be determined every second, every other second, etc. As discussed elsewhere herein, the decision rate may also be adaptable.

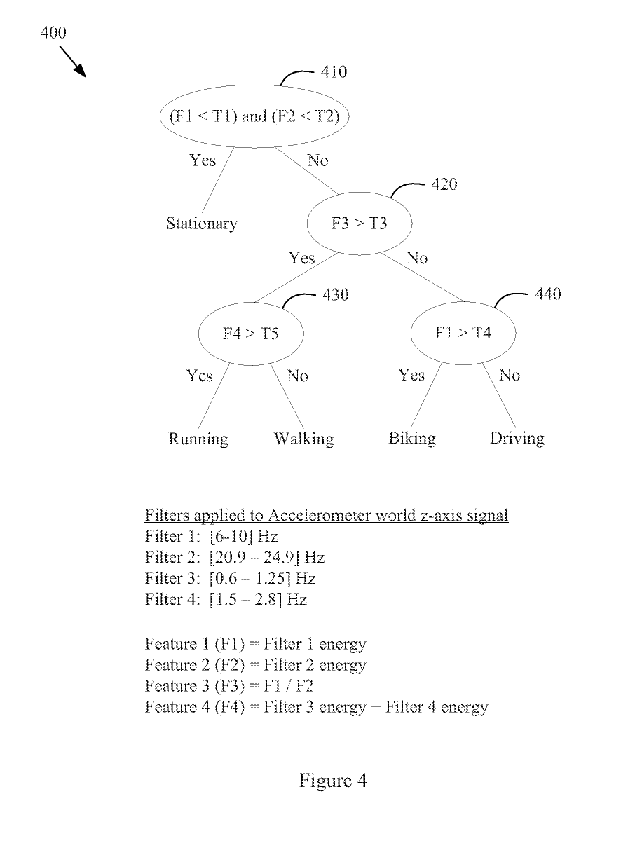

Another example of a decision tree 400, which may for example be implemented by the multiclass decision tree module 250, is presented with respect to FIG. 4.

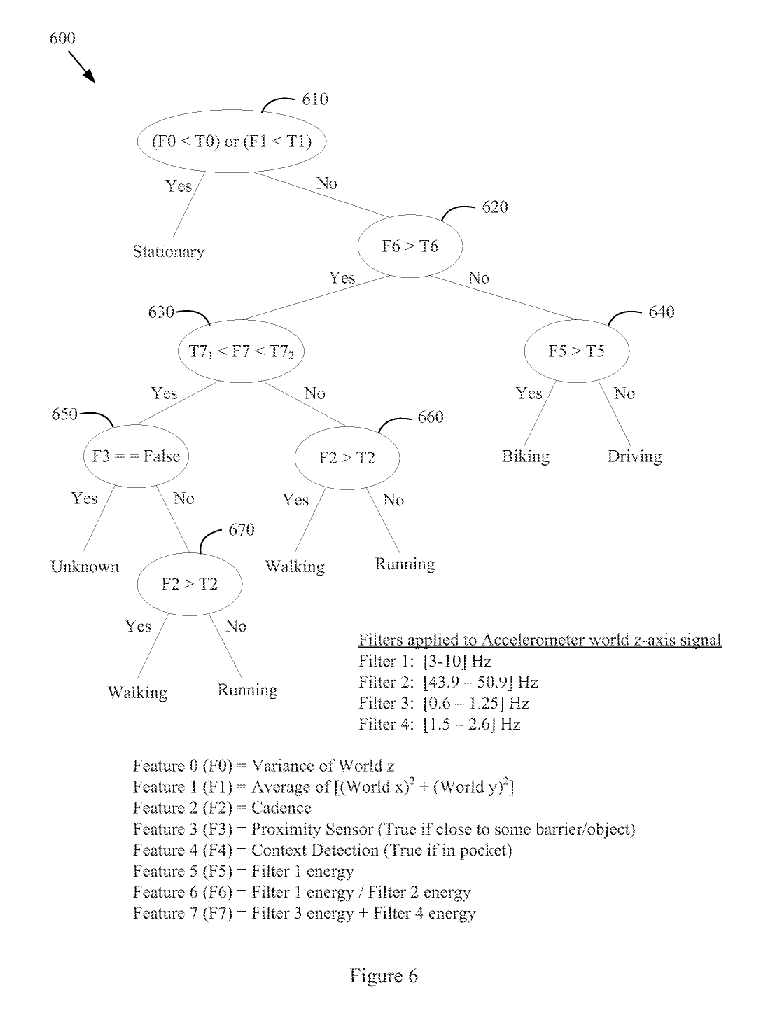

Turning now to FIG. 4, such figure shows an example multiclass decision tree 400, in accordance with various aspects of the present disclosure. The multiclass decision tree 400 is merely an example selected for illustrative clarity. Accordingly, the scope of various aspects of this disclosure should not be limited by any characteristics of the multiclass decision tree 400. The multiclass decision tree 400 may, for example, share any or all characteristics with other decision trees discussed herein (e.g., the decision tree 300 illustrated in FIG. 3 and discussed herein, the decision tree 600 illustrated in FIG. 6 and discussed herein, etc.). The multiclass decision tree 400 may, for example be utilized in an implementation of a wearable device. The multiclass decision tree 400 may also, however, be utilized in an implementation of a handheld device.

Traversal of the example decision tree 400 begins at the root node 410, at which Feature 1 (or F1) is compared to a first threshold value T1 and Feature 2 (or F2) is compared to a second threshold value T2. If, for example, Feature 1 is less than the first threshold T1 and Feature 2 is less than the second threshold T2, then traversal of the decision tree 400 proceeds to a leaf node that indicates that the device (e.g., the device 100 of FIG. 1, a device implementing the system 200 of FIG. 2, a device implementing the system 500 of FIG. 5, etc.) is associated with a stationary state, for example the device or a user thereof is determined to be in a stationary state. If, however, either Feature 1 is not less than the threshold T1 or Feature 2 is not less than the threshold T2, then traversal of the decision tree 400 proceeds to a second interior node 420, at which the activity class could be any of walking, running, biking, or driving.

At the second interior node 420, the multiclass decision tree 400 compares Feature 3 (or F3) to a threshold value T3. In this example, Feature 3 may comprise a ratio of Feature 1 to Feature 2, where Feature 1 may for example comprise an indication of energy in the Filter 1 signal (e.g., signal X1 from the filter bank 230) and Feature 2 may for example comprise an indication of energy in the Filter 2 signal (e.g., signal X2 from the filter bank 230). If, for example, Feature 3 exceeds the threshold T3, then traversal of the decision tree 400 proceeds to a third interior node 430, at which the activity class could be either walking or running. If, however, Feature 3 does not exceed the threshold T3, then traversal of the decision tree 400 proceeds to a fourth interior node 440, at which the activity class could be either biking or driving.

At the third interior node 430, the multiclass decision tree 400 compares Feature 4 (or F4) to a fifth threshold value T5. In this example, Feature 4 may comprise an indication of energy in the Filter 3 signal (e.g., signal X3 from the filter bank 230) summed with an indication of energy in the Filter 4 signal (e.g., signal X4 from the filter bank 230). If, for example, Feature 4 exceeds the fifth threshold T5, then traversal of the decision tree 400 proceeds to a leaf node that indicates that the device (e.g., the device 100 of FIG. 1, a device implementing the system 200 of FIG. 2, a device implementing the system 500 of FIG. 5, etc.) is associated with a running state, for example a user of the device is running with the device. If, however, Feature 4 does not exceed the fifth threshold T5, then traversal of the decision tree 400 proceeds to a leaf node that indicates that the device (e.g., the device 100 of FIG. 1, a device implementing the system 200 of FIG. 2, a device implementing the system 500 of FIG. 5, etc.) is associated with a walking state, for example a user of the device is walking with the device.

At the fourth interior node 440, the multiclass decision tree 400 compares Feature 1 (or F1) to a fourth threshold value T4. In this example, Feature 1 may comprise an indication of energy in the Filter 1 signal (e.g., signal X1 from the filter bank 230). If, for example, Feature 1 exceeds the fourth threshold T4, then traversal of the decision tree 400 proceeds to a leaf node that indicates that the device (e.g., the device 100 of FIG. 1, a device implementing the system 200 of FIG. 2, a device implementing the system 500 of FIG. 5, etc.) is associated with a biking state, for example a user of the device is biking with the device. If, however, Feature 1 does not exceed the fourth threshold T4, then traversal of the decision tree 400 proceeds to a leaf node that indicates that the device (e.g., the device 100 of FIG. 1, a device implementing the system 200 of FIG. 2, a device implementing the system 500 of FIG. 5, etc.) is associated with a driving state, for example a user of the device is driving or riding in a vehicle.

The features and/or thresholds utilized in the decision tree 400 may, for example, be adaptable. For example, as differences between features and thresholds become known (e.g., empirically), the features and/or thresholds may be adjusted. For example, referring to the decision tree 400 traversal through the second node 420, as operation of the device and the decision tree 400 is monitored over time, a better understanding of the differences between (running or walking) and (biking and driving) for the device or user may result in a shifting of the third threshold T3. Also for example, a better understanding of the differences between running and walking may result in a shifting of the fifth threshold T5. Additionally for example, a better understanding of the differences between biking and driving may result in a shifting of the fourth threshold T4. Additionally, user feedback may be utilized to adjust the parameters of the decision tree 400. For example, information of the selected state may be provided by the device 100 to the user, who may then provide feedback when an incorrect and/or correct state has been selected. The multiclass decision tree module 250 (or other module of the system 200) may then identify the node at which the incorrect decision was made and adjust the feature and/or threshold parameters at such node to reduce the chance that the same error will occur again. In such a manner, operation of the decision tree 400 may be tailored to a device and/or user thereof.

The activity class decisions may be performed at any of a variety of rates. The decision rate may, for example, correspond to the sensor update rate, a step rate, regular timed intervals, etc. The decision may, for example, be performed continually, but sampled at another rate. For example, the activity class may be determined every second, every other second, etc. As discussed elsewhere herein, the decision rate may also be adaptable.

Returning back to FIG. 2, the multiclass decision tree module 250 may output its determined activity class to the state transition module 260. The state transition module 260 may, for example, analyze the activity class determined by the multiclass decision tree module 250 to reach a final activity class decision.

For example, the detected activity class output by the multiclass decision tree module 250 may, for example, change when a user has just temporarily modified the activity. For example, a walking or running user might stop to wait for traffic, a biker might stop paddling, an automobile might stop, etc. In other words, temporary changes in user behavior might be seen as a significant change in user activity, when in fact the change is just temporary.

Accordingly, various aspects of the system include a state transition module 260 that may, for example, operate to eliminate unnecessary jitter in detected activity. For example, if the activity states detected by the multiclass decision tree module 250 are: walk, walk, walk, walk, walk, walk, stationary, stationary, walk, and walk, the state transition module 260 may analyze the sequence of detected activity states to determine that the user was generally engaged in walking the entire time. As an example, the state transition module 260 may operate such that a walking user must be detected stationary by the multiclass decision tree module 250 for a particular amount of time or for a particular number of activity class determinations before a change in detected activity state from walking to stationary is recognized (or finalized) and output.

The decision criteria utilized by the state transition module 260 may, for example, be adaptive. For example, if a user has just started walking or driving or has been in such state(s) for a relatively small amount of time, it may take less time or fewer activity class decisions to be declared stationary than if a user has been engaged in walking or driving for a substantially longer period of time. For example, a series of detected activity states of [stationary, walk, walk, stationary] may be enough to be declared stationary, but a series of 10 walk determinations followed by a single stationary determination might still result in an overall determination of walking, albeit at a lower confidence level than before the stationary determination.

Additionally for example, particular activity classifications may have different respective activity change criteria. For example, while walking can occur for short, medium, and long durations, most biking or driving occurs over medium and long durations. Thus, the criteria for exiting the biking or driving states may be relatively more stringent than the criteria for exiting a walking state. Such different criteria may, for example, be reflected in the state transition module 260 (e.g., in a number of changed states needed to be deemed a valid changed state) and/or in the multiclass decision tree module 250 (e.g., in an adjusted threshold) and/or in the feature computation module 240 (e.g., in an adjusted feature computation).

Further for example, the state transition module 260 may consider different respective time windows of activity class determinations from the decision tree for different respective activity classes. For example, for driving, the state transition module may analyze a 15-second window of activity classes detected by the multiclass decision tree module 250, while for walking, the state transition module 260 may analyze a 5-second window of detected activity classes.

The selection criteria may, for example, also change over time based on monitored user behavioral patterns. For example, if a user generally bikes to and from work during particular times of day, the state transition module 260 may use information of such an activity pattern combined with day/time information to readily enter and/or exit the biking state at the habitual times. In such an example, a clock and/or calendar may be viewed as a non-limiting example of a passive sensor. Also for example, if a user generally runs on a particular route, rather than walking, biking or driving, then the state transition module 260 may use information of such an activity pattern combined with location information to readily enter and/or exit the running state at the habitual times. In such an example, a collection of information of past history may be view as another non-limiting example of a passive sensor. Other examples of passive sensors (or passive sensor information) may, for example, comprise pedometer cadence and/or speed, email, user input, calorie burn information, map information, etc.

The selection criteria may also, for example, be based on user preferences input into the system 200. For example, a user that does not own a car may provide an input that causes the state transition module 260 to have a high threshold (or other strict criteria) for detecting a “driving” activity. Also for example, a user that bikes often may provide an input that causes the state transition module 260 to have relatively low threshold (or light criteria) for classifying the user's activity as “biking.” Additionally for example, the system 200 may also accept a user input that exactly specifies the present activity in which the user is engaged. For example, a user may tell the system 200 that the user is going for a bike ride. In such a scenario, the system 200 may adopt the user's specified activity always, or may adopt the user's specified activity with a high emphasis (e.g., still looking for criteria indicating that the user's activity has changed). As mentioned elsewhere herein, when an activity is known, one or more modules of the system 200 may operate to analyze the signals received to determine whether an adjustment to threshold, feature determinations, etc., is warranted.

The selection criteria may also, for example, change depending on the number of activity classes that are available for selection. For example, in general, the more classes that are available for selection, the greater the opportunity is for confusion between the classes. In such an environment, a high number of available classes for selection may warrant a slower filter for changing an activity class (e.g., looking at more values). In such a scenario, a user may also be provided with an interface by which the user may eliminate activity classes in which the user never or rarely engages. For example, a user may eliminate biking from the list of selectable activity classes if the user never bikes. In such a scenario, the operation of any or all of the modules discussed herein may be modified to adapt to the modified list of selectable activity classes. For example, the multiclass decision tree module 250 may be capable of operating in accordance with a plurality of different decision tree models, where the present tree is selected based on the defined set of activity classes. For example, rather than just removing a user-specified activity class from a decision tree that was designed to include the user-specified activity class, an entirely different decision tree that was designed and/or optimized without the user-specified activity class may be utilized.

The state transition module 260 may, for example, output a “final” activity class selection. The state transition module 260 may, for example, communicate the final activity class selection to a host (or application) processor, which may then for example operate the device accordingly.

Various aspects of this disclosure may also comprise tilt determination. For example, for various devices (e.g., a mobile phone), the host might want to know whether the device has been picked up by the user. For example, if the angle of the phone has changed, but not during walking, running biking, driving, etc., the system 200 may determine that the device has been picked up for operation by the user.

In an example implementation, the sensor fusion module 210 may generate an orientation and/or a rotation matrix for transforming a location or vector in the body coordinate system to the world (or inertial) coordinate system. The orientation and/or rotation matrix may, for example, be expressed in quaternion-related coefficients, but may alternatively be expressed in Euler angle-related coefficients.

The orientation and/or rotation matrix, or various coefficients thereof, may be output from the sensor fusion module 210 to a tilt angle difference (TAD) determination module 270. The TAD determination module 270 may then, for example, identify the extent to which a tilt angle of the device has changed relative to a reference tilt angle (e.g., relative to a most recent steady state tilt, a world coordinate reference vector, etc.).

As mentioned above, in various scenarios, the tilt angle may be more interesting when the “stationary” activity class has been detected (e.g., a non-stationary activity class such as running, walking, driving, biking, etc., has not been detected). Thus, the tilt decision module 280 may receive the calculated tilt angle and/or tilt angle change from the TAD determination module 270 and receive the final activity class from the state transition module 260, and base its decision of whether a significant tilt has occurred at least in part on such parameters.

For example, if the tilt angle difference (TAD) is greater than a threshold, and the user's activity class has been identified as stationary (or not walking, running, biking, driving, etc.), the tilt decision module 280 may generate and output a “tilt” signal. Such a tilt signal may, for example, notify the host (or application) processor that the user has just picked up or taken out the device.

Various additional aspects of the disclosure may comprise motion determination aspects. For example, the state transition module 260 may output an indication of whether a significant amount of motion (or user movement) has occurred. Information of detected motion, regardless of the source (e.g., biking, walking, running, driving, etc.), may for example be utilized to trigger location services and/or other services, which can otherwise be shut down when the user is not in motion. The state transition module 260 may base such a determination, at least in part, on the specific activity class identified. The state transition module 260 may also, for example, use different selection criteria than used for identifying the particular activity class.

The example system 200 is presented to illustrate various aspects of the disclosure. Reference will now be made to FIG. 5, which shows another example system 500 to illustrate various aspects of the disclosure. It should be understood that any or all of the aspects of the example systems 100, 200 and 500 may be combined into a single system.

FIG. 5 shows an example activity classification system 500 in accordance with various aspects of the present disclosure. The example system 500 may, for example, be used to classify an activity of a user using a handheld device (e.g., a mobile telephone, PDA, camera, portable media player, gaming device, etc.). Note, however, that the system 500 is not limited to handheld devices, for example being readily applicable to wearable devices (e.g., a watch, a headband, an armband, a belt-mounted device, eyeglasses, etc.) and other devices. The system 500 shown in FIG. 5 may share any or all characteristics with the system 100 shown in FIG. 1 and discussed herein. For example, the system 500 may, for example, be implemented with the sensor processor 130 of FIG. 1 operating in accordance with software instructions in the sensor fusion software module 144 and activity classification software module 142 stored in the internal memory 140. Also for example, the system 400 or any portion thereof may be implemented with the application (or host) processor 112 operating in accordance with software instructions stored in the application memory 114. The system 500 may also, for example, share any or all characteristics with the example system 200 shown in FIG. 2 and discussed herein.

The discussion of FIGS. 1-4 focused generally on processing the signal A_{w}^{z}, but as explained herein, the various aspects of this disclosure are not limited thereby. For example, other coefficients of the accelerometer vector may be analyzed and/or one or more coefficients of the gyroscope vector may also be analyzed. For example, devices (e.g., handheld devices, wearable devices, etc.) may experience motions due to hand movements that are not associated with an activity of interest. As an example, a hand motion performed while strumming a guitar may be confused with a hand swinging motion due to pedestrian activity like jogging. As another example, driving a car (e.g., hands on wheel) is associated with different forces with a device worn on the wrist than with a handheld device, which is typically set down, pocketed or cradled during driving.

To gain additional insight into the user's activity, various aspects of this disclosure may include looking at other signals in addition to the signal A_{w}^{z}. The accelerometer vector and/or the gyroscope vector may, for example, be input to the sensor fusion module 510, which transforms such signals into the world (or inertial) coordinate system. The output vectors are labeled A_{w} and G_{w}. The sensor fusion module 510 may, for example, share any or all characteristics with the sensor fusion module 210 of the system 200 illustrated in FIG. 2 and discussed herein.

The signals A_{w} and G_{w} may, for example, be viewed as discrete time signals that are updated at a sensor update rate of the accelerometer signal and/or the gyroscope signal. Note, however, any of a variety of sensor signal update and/or activity class determination rates may be utilized. For example, the respective update rates of such signals may be the same or different. The calculations and determinations herein may also be performed at a sensor rate. Note, however, as explained herein any of a variety of sensor signal update and/or activity determination rates may be utilized.

From the sensor fusion module 510, the signals A_{w} and G_{w} may, for example, be provided to a signal conditioning module 520 to prepare the A_{w} and G_{w} signals for subsequent processing. The signal conditioning module 520 may, for example, remove noise from the A_{w} and G_{w} signals, restore shape to clipped A_{w} and/or G_{w} signals, adjust amplitude of the A_{w} and/or G_{w} signals (e.g., removing a known bias, scaling the signal for subsequent processing, etc.), etc. The signal conditioning module 520 may, for example, share any or all characteristics with the signal condition module 220 of the system 200 illustrated in FIG. 2 and discussed herein.

The conditioned A_{w} and G_{w} signals may then be provided to a filter bank module 530. As mentioned herein, the spectral content of the A_{w} and G_{w} signals may change based on user activity. The filter bank module 530 may, for example, comprise a plurality of filters that operate to isolate frequency ranges of interest in the A_{w} signal and/or in the G_{w} signal. The example filter bank module 530 is illustrated with four filters, but may comprise any number of filters. The example filter bank module 530 may, for example, share any or all characteristics with the example filter bank module 230 of the system 200 shown in FIG. 2 and discussed herein. The example filter bank module 530 may utilize the same filters as discussed with regard to the filter bank module 230 of FIG. 2 and/or with regard to the example decision trees 300 and 400 of FIGS. 3 and 4, but may also use different filters. For example, example filters may comprise bandpass filters with the following corner frequencies:
Filter 1 (X1): 3 to 10 Hz
Filter 2 (X2): 43.9 to 50.9 Hz
Filter 3 (X3): 0.6 to 1.25 Hz
Filter 4 (X4): 1.5 to 2.6 Hz

The filter characteristics are merely examples. The example filter characteristics were found empirically to work well, but the scope of the disclosure is not limited to the number of filters nor by the respective characteristics of each of such filters. The number of filters and/or characteristics of the filters may change depending on device type, depending on the user, depending on real-time activity indicated by sensor signals or other device signals, etc. Other non-limiting filter bank examples are provided herein.

The filtered signal outputs from the filter bank 530 (e.g., Filter 1 to Filter 4), labeled X1-X4, are provided to a feature computation module 540. The filtered signal output from the filter bank 530 may, for example, correspond to filtered or unfiltered accelerometer signals and/or filtered or unfiltered gyroscope signals.

The feature computation module 540 may, for example, process the inputs X1-X4 (e.g., individually and/or in any combination) to identify features of interest in the filtered signals. The feature computation module 540 may, for example, share any or all characteristics with the feature computation module 240 shown in FIG. 2 and discussed herein. As discussed with regard to FIG. 2, the feature computation module 540 may, for example, determine and/or estimate the respective energy found in each of the input signals X1-X4 (e.g., signal amplitudes, squared signal amplitudes, signal amplitudes integrated over time, etc.). Non-limiting examples of such determination and/or estimation are provided herein (e.g., with regard to the feature computation module 240 of FIG. 2). Also for example, as discussed with regard to FIG. 2, a variability computation may be incorporated (e.g., with regard to accelerometer and/or gyroscope signals).

As mentioned herein, feature computation module 540 may process inputs received from any of a variety of sources, non-limiting examples of which are shown in FIG. 5. For example, the feature computation module 540 may receive information from a pedometer. Such a pedometer may, for example, be local to the system 500 and/or external to the system 500. For example, the feature computation module 540 may receive information of user cadence (e.g., stepping rate or frequency) from the pedometer. Also for example, the feature computation module 540 may receive context information from the pedometer or other circuit (e.g., information of whether a device utilizing the system 500 is positioned in a user's pocket). In such an example, pedometer cadence and/or speed information may be viewed as a non-limiting example of passive sensor information.

Additionally, the feature computation module 540 may process inputs received from a proximity sensor, for example, before or after proximity sensor signals are processed by a signal conditioning module 570. A proximity sensor may, for example, determine whether a device implementing the system 500 is close to some object, barrier, etc.

The feature computation module 540 may also combine features, for example features of individual filtered signals, features associated with different respective sensors and/or information sources, etc., to calculate more complex features. For example, the feature computation module 540 may calculate a feature as a sum and/or ratio of other features. For example, a set of features may comprise:
Feature 1 (F1): Average of [(A_{w}^{x})²+(A_{w}^{y})²]
Feature 2 (F2): Cadence
Feature 3 (F3): Proximity Sensor (True if close to some barrier/object)
Feature 4 (F4): Context Detection (True if in pocket)
Feature 5 (F5): Filter 1 energy
Feature 6 (F6): Filter 1 energy/Filter 2 energy
Feature 7 (F7): Filter 3 energy+Filter 4 energy

Additionally for example, in various implementations, the feature computation module 540 may determine (or compute) one or more indications of variability (e.g., of the filtered signals, of combinations of the filtered signals, etc.). For example, the feature computation module 540 may determine the variance (or standard deviation) of the A_{w}^{z} signal (e.g., after signal conditioning as shown in FIG. 5, or before signal conditioning). For example, continuing the example above:
Feature 0 (F0): Variance of A_{w}^{z}

The feature computation module 540 may output the determined features (e.g., metrics or characteristics), which may then for example be provided to a multiclass decision tree module 550. The multiclass decision tree module 550 may, for example, analyze the features received from the feature computation module 540 to identify an activity class.

Decision trees are discussed herein, and examples are provided with respect to FIGS. 3 and 4. Another example of a decision tree 500, which may for example be implemented by the multiclass decision tree module 550, is presented with respect to FIG. 6.

Turning now to FIG. 6, such figure shows an example multiclass decision tree 600, in accordance with various aspects of the present disclosure. The multiclass decision tree 600 is merely an example selected for illustrative clarity. Accordingly, the scope of various aspects of this disclosure should not be limited by any characteristics of the multiclass decision tree 600. The multiclass decision tree 600 may, for example, share any or all characteristics with other decision trees discussed herein (e.g., the decision tree 300 illustrated in FIG. 3 and discussed herein, the decision tree 400 illustrated in FIG. 4 and discussed herein, etc.). The multiclass decision tree 600 may, for example be utilized in an implementation of a handheld device. The multiclass decision tree 600 may also, however, be utilized in an implementation of a wearable device or other type of device.

Traversal of the decision tree 600 begins at the root node 610, at which Feature 0 (or F0) is compared to a threshold value T0 and Feature 1 (or F1) is compared to a first threshold value T1. In this example, Feature 0 may comprise an indication of variance of the A_{w}^{z} signal, and Feature 1 may comprise an average value of the sum of respective squares of the A_{w}^{x} and A_{w}^{y} signals. If, for example, Feature 0 is less than the threshold T0, and/or Feature 1 is less than the first threshold T1, then traversal of the decision tree 600 proceeds to a leaf node that indicates that the device (e.g., the device 100 of FIG. 1, a device implementing the system 200 of FIG. 2, a device implementing the system 500 of FIG. 5, etc.) is associated with a stationary state, for example the device or a user thereof is determined to be in a stationary state. If, however, Feature 0 is not less than the threshold T0 and Feature 1 is not less than the first threshold T1, then traversal of the decision tree 600 proceeds to a second interior node 620, at which the activity class could be any of walking, running, biking, and/or driving.

At the second interior node 620, the multiclass decision tree 600 compares Feature 6 (or F6) to a sixth threshold value T6. In this example, Feature 6 may comprise a ratio of a respective indication of energy in the Filter 1 signal (e.g., signal X1 from the filter bank 530) to a respective indication of energy in the Filter 2 signal (e.g., signal X2 from the filter bank 530). If, for example, Feature 6 exceeds the sixth threshold T6, then traversal of the decision tree 600 proceeds to a third interior node 630, at which the activity class could be running or walking or an unknown activity. If, however, Feature 6 does not exceed the sixth threshold T6, then traversal of the decision tree 600 proceeds to a fourth interior node 640, at which the activity class could be either biking or driving.

At the third interior node 630, the multiclass decision tree 600 compares Feature 7 (or F7) to a lower seventh threshold T7₁ and an upper seventh threshold T7₂. In this example, Feature 7 may comprise a sum of a respective indication of energy in the Filter 3 signal (e.g., signal X3 from the filter bank 530) and a respective indication of energy in the Filter 4 signal (e.g., signal X4 from the filter bank 530). If, for example, Feature 7 is between the lower seventh threshold T7₁ and the upper seventh threshold T7₂, then traversal of the decision tree 600 proceeds to a fifth interior node 650, at which the activity class could be running or walking or an unknown activity. If, however, Feature 7 is not between the lower seventh threshold T7₁ and the upper seventh threshold T7₂, then traversal of the decision tree 600 proceeds to a sixth interior node 660, at which the activity class could be running or walking

At the fifth interior node 650, the multiclass decision tree 600 compares Feature 3 (F3) to a logical False value. In this example, F3 may comprise an indication of whether the device implementing the system 500 is close to an object or barrier. For example, F3 may have a logical True value if the device is close to an object or barrier. If, for example, Feature 3 is logical False, then traversal of the decision tree 600 proceeds to a leaf node that indicates that the device (e.g., the device 100 of FIG. 1, a device implementing the system 200 of FIG. 2, a device implementing the system 500 of FIG. 5, etc.) is associated with an unknown activity. If, however, Feature 3 is not logical False, then traversal of the decision tree 600 proceeds to a seventh interior node 670, at which the activity class could be walking or running

At the seventh interior node 670, the multiclass decision tree 600 compares Feature 2 (F2) to a second threshold T2. In this example, F2 may comprise an indication of cadence. If, for example, Feature 2 is greater than the second threshold T2, then traversal of the decision tree 600 proceeds to a leaf node that indicates that the device (e.g., the device 100 of FIG. 1, a device implementing the system 200 of FIG. 2, a device implementing the system 500 of FIG. 5, etc.) is associated with a walking state (e.g., the device is being carried by a user that is walking) If, however, Feature 2 is not greater than the second threshold T2, then traversal of the decision tree 600 proceeds to a leaf node that indicates that the device (e.g., the device 100 of FIG. 1, a device implementing the system 200 of FIG. 2, a device implementing the system 500 of FIG. 5, etc.) is associated with a running state (e.g., the device is being carried by a user that is running)

At the sixth interior node 660, the multiclass decision tree 600 compares Feature 2 (F2) to the second threshold T2. If, for example, Feature 2 is greater than the second threshold T2, then traversal of the decision tree 600 proceeds to a leaf node that indicates that the device (e.g., the device 100 of FIG. 1, a device implementing the system 200 of FIG. 2, a device implementing the system 500 of FIG. 5, etc.) is associated with a walking state, for example a user of the device is walking with the device. If, however, Feature 2 is not greater than the second threshold T2, then traversal of the decision tree 600 proceeds to a leaf node that indicates that the device (e.g., the device 100 of FIG. 1, a device implementing the system 200 of FIG. 2, a device implementing the system 500 of FIG. 5, etc.) is associated with a running state, for example a user of the device is running with the device.

At the fourth interior node 640, the multiclass decision tree 600 compares Feature 5 (F5) to a fifth threshold T5. In this example, F5 may comprise an indication of energy in the Filter 1 signal (e.g., signal X1 from the filter bank 530). If, for example, Feature 5 is greater than the fifth threshold T5, then traversal of the decision tree 600 proceeds to a leaf node that indicates that the device (e.g., the device 100 of FIG. 1, a device implementing the system 200 of FIG. 2, a device implementing the system 500 of FIG. 5, etc.) is associated with a biking state (e.g., the device is being carried by a user that is biking) If, however, Feature 5 is not greater than the fifth threshold T5, then traversal of the decision tree 600 proceeds to a leaf node that indicates that the device (e.g., the device 100 of FIG. 1, a device implementing the system 200 of FIG. 2, a device implementing the system 500 of FIG. 5, etc.) is associated with a driving state (e.g., the device and/or user thereof is riding in a car).

The features and/or thresholds utilized in the decision tree 600 may, for example, be adaptable. For example, as differences between features and thresholds become known (e.g., empirically), the features and/or thresholds may be adjusted. For example, referring to the decision tree 600 traversal through the second interior node 620, as operation of the device and the decision tree 600 is monitored over time, a better understanding of the differences between (running or walking) and (biking or driving) for the device or user may result in a shifting of the sixth threshold T6. Also for example, a better understanding of the differences between running and walking may result in a shifting of the second threshold T2. Additionally for example, a better understanding of the differences between biking and driving may result of a shifting of the fifth threshold T5. Additionally, user feedback may be utilized to adjust the parameters of the decision tree 600. For example, information of the selected state may be provided by the device 100 to the user, who may then provide feedback when an incorrect and/or correct state has been selected. The multiclass decision tree module 550 (or other module of the system 500) may then identify the node at which the incorrect decision was made and adjust the feature and/or threshold parameters at such node to reduce the chance that the same error will occur again. In such a manner, operation of the decision tree 600 may be tailored to a device and/or user thereof.

The activity class decisions may be performed at any of a variety of rates. The decision rate may, for example, correspond to the sensor update rate, a step rate, regular timed intervals, etc. The decision may, for example, be performed continually, but sampled at another rate. For example, the activity class may be determined every second, every other second, etc. As discussed elsewhere herein, the decision rate may also be adaptable.

Returning back to FIG. 5, the multiclass decision tree module 550 may output its determined activity class to the state transition module 560. The state transition module 560 may, for example, analyze the activity class determined by the multiclass decision tree module 550 to reach a final activity class decision. The state transition module 560 may, for example, share any or all characteristics with the state transition module 260 shown in FIG. 2 and discussed herein.

As discussed herein, the state transition module 560 may, for example, operate to eliminate unnecessary jitter in detected activity states. The decision criteria may, for example, be adaptive, and particular activity classifications may have different respective activity change criteria. The state transition module 560 may look at different respective time windows of activity determinations from the multiclass decision tree module 550 for different respective activities. The selection criteria may also change over time based on monitored user behavioral patterns. The selection criteria may also, for example, be based on user preferences input into the system 500. The selection criteria may additionally, for example, change depending on the number of activity classes that are available for selection.

The state transition module 560 may, for example, output a “final” activity class selection. The state transition module 560 may, for example, communicate the final activity class selection to a host (or application) processor, which may then for example modify operation of the device accordingly.

As discussed herein, information from sensors different from the accelerometer and/or gyroscope may also be integrated into the system. Some of examples of such information and sensors have already been provided herein. Additional examples will now be discussed.

For example, microphone information may be considered. Particular audio content may be associated with respective user activities. For example, audio processing may detect the presence of engine noise, tire noise, wind noise, walking cadence, peddling cadence, running or walking cadence, biometric information like pulse rate and breathing rate, environmental background noise, shock and/or vibration noise, etc. The system illustrated in FIG. 5 shows a microphone signal being conditioned at an audio signal conditioning module 582, filtered at an audio filter bank 584, and provided to the feature computation module 540. The feature computation module 540 may, for example, determine overall sound energy, sound energy in particular frequency bands (e.g., those associated with activity classes of interest), etc. The feature computation module 540 may, for example, analyze time domain zero crossings and/or amplitudes.

The multiclass decision tree (e.g., examples of which are provided with respect to FIGS. 3, 4, and 6) may then, for example, comprise node transitions that are based, at least in part, on audio information.

The utilization of audio information may, for example, provide for the detection of additional activity classes and/or sub-classes. For example, walking or running on a treadmill will have a different sound signature (e.g., including motor and belt noise and less wind noise) than walking or running on a sidewalk or trail. Also for example, biking on a stationary bike will have a different sound signature (e.g., including motor and belt noise and less wind noise and less road noise and different vibrations) than biking outside.

Various aspects of the disclosure may comprise the incorporation of pressure sensor information into the activity classification analysis. For example, pressure sensor information can be utilized to ascertain whether a user is climbing while walking (e.g., up stairs, on a trail, etc.). Also for example pressure sensor information can be utilized to ascertain an elevation at which the user is active, whether the user is flying based at least in part on cabin pressure, etc. FIG. 5 illustrates an example pressure sensor input to a pressure signal conditioning module 592 and a pressure filter bank module 594 (e.g., comprising one or more filters). The system 500 may thus isolate the pressure sensor information of interest for the feature computation module 540 and/or multiclass decision tree module 550.

Various aspects of the disclosure may comprise the incorporation of magnetometer information into the activity classification analysis. For example, magnetometer information may be utilized to ascertain whether a user is riding a subway and/or train. Also for example, magnetometer information can be utilized to ascertain a user's location and/or movement in relation to known man-made and/or natural magnetic field sources, which may for example, be mapped. In another example, the user's automobile (e.g., an electric vehicle, hybrid vehicle, golf cart, etc.) may have a particular magnetic field signature that is recognizable. In a further example, a user's place of employment (e.g., office, manufacturing facility, distribution facility, etc.) may have a particular magnetic field signature that is recognizable. FIG. 5 illustrates an example magnetometer sensor input to a magnetometer signal condition module 598

Various aspects of the disclosure may comprise the incorporation of location information into the activity classification analysis. For example, information of a user's location may be helpful when determining what a user is doing. For example, a user on a mountain bike trail will likely be mountain biking, a user on a hiking trail will likely be hiking, a user in an office will likely be walking when moving, a user in a shopping mall will likely be walking when moving, a user on a highway will likely be driving, etc. Additionally, speed or velocity information may be useful to distinguish between biking on a road and driving on a road, between walking and running, etc. FIG. 5 illustrates an example GPS (or other location system) input to a location signal conditioning module 596. The system 500 may thus condition the location information of interest for the feature computation module 540 and/or multiclass decision tree module 550.

Access to location information may also help to identify various sporting classes. For example, location on a tennis court, a basketball court, a fitness center weight room, yoga studio, dojo, etc. may provide valuable insight into the user's activity. For example, a running and/or walking determination may otherwise be classified as playing tennis, playing golf, walking the beach, household work, etc. Also for example, a biking determination may otherwise classified as mountain biking, road biking, hill climbing, etc.

Various aspects of the disclosure may further comprise the incorporation of biometric information into the activity classification analysis. For example, heart rate information may assist in distinguishing between running, walking, walking uphill, etc. Similarly, body temperature information may be utilized. Biometric sensors may, for example, provide for the determination of sleep activity.

Various aspects of the disclosure may further comprise the incorporation of ambient temperature and/or humidity sensors in the activity classification analysis. For example, temperature information may help distinguish between indoor (or in-car) and outdoor activity. Also for example, humidity information may help distinguish between lifting weights and performing less strenuous movements.

Various aspects of the disclosure may further comprise the incorporation of wireless signal information (e.g., Bluetooth signal, Wi-Fi signal, cellular signal, UWB signal, etc.) into the activity classification analysis. For example, proximity to various signal sources (e.g., an automobile Bluetooth signal) may assist in distinguishing between riding in a car and biking. Also for example, continued proximity to a home Wi-Fi network may assist in distinguishing between doing housework and yard work. Further for example, detection of a shopping mall Wi-Fi network may assist in distinguishing between walking and running Additionally for example, detection of a fitness center Wi-Fi signal may assist in distinguishing between weight lifting and general arm movement.

Other sensors (e.g., non-inertial) may augment the information provided by inertial sensors, but may also replace inertial and/or other sensor information. Example non-inertial sensors may, for example, comprise a magnetometer, microphone, any of a variety of biometric sensors, thermometers, pressure sensors, proximity sensor, moisture sensors, light sensors, proximity sensors, etc. For example, in a scenario in which analysis of an audio signature determines with confidence that the user is driving, the system need not analyze information from other sensors and thus save energy. Also for example, in a scenario in which analysis of a biometric signal indicates that the user is sleeping, the system 500 need not analyze information from other sensors. Additionally for example, in a scenario in which analysis of an audio signal determines with confidence that the user is walking, running or biking, the system 500 need not analyze information from other sensors. Further for example, in a scenario in which analysis of a magnetometer signal provides an indication that a user is traveling in a subway, the system 500 need not analyze information from other sensors. Accordingly, various aspects of this disclosure provide for selecting between sensors.

Various aspects of this disclosure may comprise prioritizing sensors to be analyzed. Such prioritization may, for example, be based on expected energy consumption. For example, though as explained above location information may be advantageous in many ways, such information is often associated with relatively high energy consumption. Thus, to save energy, various aspects of this disclosure may comprise resorting to location information only when the activity classification cannot be performed to at least a particular degree of confidence using other sensors. For example, various sensors may be turned on or off based on need. For example, in an example scenario, relatively high-power sensors may be turned off or otherwise operated in a low-power mode unless an activity classification decision cannot be adequately made without the utilization of such sensors. In such a scenario, a decision may then be made to turn on or otherwise activate the desired sensor(s) when the need arises. Whether various sensors should be turned on or off or placed in a low-power mode may be determined based on any or a variety of criteria, for example based on a factory-defined priority, based on user input, based on past activity class determination, based on a presently identified activity class, based on time and/or date, based on geographical location, based on a next anticipated activity class, etc.

Various aspects of this disclosure may comprise outputting an indication of confidence associated with one or more activity class identifications. For example, as discussed herein, activity classes may have temporary interruptions, for example stopping while walking. If a walking user has stopped, the system 500 may determine that the user is likely still walking but has temporarily stopped. In such a case, the system 500 may identify the walking activity class, but with a degree of uncertainty that grows with each indication of stationary. For example, the system 500 may identify the walking activity with an 80% certainty level. The system 500 may also assign and/or identify a 20% chance that the user has stopped walking and is now stationary. Such information may, for example, provide a host system with information it needs to make higher-level operational decisions.

The systems illustrated in FIGS. 1-6 and discussed herein, were presented to illustrate various aspects of the disclosure. Any of the systems presented herein may share any or all characteristics with any of the other systems presented herein. Additionally, it should be understood that the various modules were separated out for the purpose of illustrative clarity, and that the scope of various aspects of this disclosure should not be limited by arbitrary boundaries between modules. For example, any one or more of the modules may share hardware and/or software with any one or more other modules.

As discussed herein, any one or more of the modules and/or functions discussed herein may be implemented by a pure hardware solution or by a processor (e.g., an application or host processor, a sensor processor, etc.) executing software instructions. Similarly, other embodiments may comprise or provide a non-transitory computer readable medium and/or storage medium, and/or a non-transitory machine readable medium and/or storage medium, having stored thereon, a machine code and/or a computer program having at least one code section executable by a machine and/or a computer (or processor), thereby causing the machine and/or computer to perform the methods as described herein.

In summary, various aspects of the present disclosure provide a system and method for efficiently and reliably classifying user activity. While the foregoing has been described with reference to certain aspects and embodiments, it will be understood by those skilled in the art that various changes may be made and equivalents may be substituted without departing from the scope of the disclosure. In addition, many modifications may be made to adapt a particular situation or material to the teachings of the disclosure without departing from its scope. Therefore, it is intended that the disclosure not be limited to the particular embodiment(s) disclosed, but that the disclosure will include all embodiments falling within the scope of the appended claims.

What is claimed is:

1. A system for activity classification, the system comprising:
at least one module operable to, at least:
receive a sensor signal;
form a plurality of filtered sensor signals by, at least in part, filtering the received sensor signal with a plurality of respective band-pass filters; and
identify an activity class based, at least in part, on the plurality of filtered sensor signals.

2. The system of claim 1, wherein the received sensor signal is expressed in a world coordinate system.

3. The system of claim 2, wherein the received sensor signal comprises a z-axis component of an accelerometer sensor signal expressed in the world coordinate system.

4. The system of claim 1, wherein the at least one module is operable to form at least four filtered sensor signals by, filtering the received sensor signal with at least four respective band-pass filters.

5. The system of claim 1, wherein the at least one module is operable to adapt the characteristics of the plurality of respective band-pass filters.

6. The system of claim 1, wherein the at least one module is operable to identify an activity class by, at least in part, determining a respective indication of energy for each of the plurality of filtered sensor signals.

7. The system of claim 6, wherein the at least one module is operable to identify an activity class by, at least in part, determining a respective running scaled sum for each of the respective indications of energy.

8. The system of claim 6, wherein the at least one module is operable to identify an activity class based, at least in part, on an immediately prior identified activity.

9. The system of claim 6, wherein the at least one module is operable to identify an activity class by, at least in part, utilizing a decision tree in which the respective indications of energy for each of the plurality of filtered sensor signals are analyzed to traverse the decision tree.

10. The system of claim 9, wherein the at least one module is operable to adapt decision criteria in the decision tree based, at least in part, on monitored user behavior.

11. The system of claim 1, wherein the at least one module is operable to identify an activity class by, at least in part, identifying the activity class at a variable rate.

12. The system of claim 1, wherein the at least one module is operable to:
determine an indication of the variability of an accelerometer signal and/or another signal derived therefrom; and
identify the activity further based, at least in part, on the determined indication of variability.

13. The system of claim 1, wherein the at least one module is operable to condition the received sensor signal to restore signal shape.

14. The system of claim 1, wherein the at least one module is operable to determine a confidence level associated with the identified activity class.

15. The system of claim 1, wherein the at least one module is operable to provide an interface by which a user of the system can define at least a portion of a set of activity classes from which the identified activity class is selected.

16. The system of claim 1, wherein the at least one module is operable to:
receive location information; and
identify the activity further based, at least in part, on the received location information.

17. The system of claim 1, wherein the at least one module is operable to:
receive pedometer information; and
identify the activity further based, at least in part, on the received pedometer information.

18. The system of claim 1, wherein the at least one module is operable to:
receive cadence information; and
identify the activity further based, at least in part, on the received cadence information.

19. The system of claim 1, wherein the at least one module is operable to:
receive proximity sensor information; and
identify the activity further based, at least in part, on the received proximity sensor information.

20. The system of claim 1, wherein the at least one module is operable to:
receive magnetometer information; and
identify the activity further based, at least in part, on the received magnetometer information.

21. A system for activity classification, the system comprising:
at least one module operable to, at least:
receive an inertial sensor signal from an inertial sensor;
receive a non-inertial sensor signal from a non-inertial sensor;
form a plurality of filtered inertial sensor signals by, at least in part, filtering the received inertial sensor signal with a plurality of respective band-pass filters; and
identify an activity class based, at least in part, on the plurality of filtered inertial sensor signals and the non-inertial sensor signal.

22. The system of claim 21, wherein the non-inertial sensor signal comprises a proximity sensor signal.

23. The system of claim 21, wherein the non-inertial sensor signal comprises a pressure sensor signal.

24. The system of claim 21, wherein the non-inertial sensor signal comprises a microphone signal.

25. The system of claim 21, wherein the non-inertial sensor signal comprises a magnetometer signal.

26. A system for activity classification, the system comprising:
at least one module operable to, at least:
receive a sensor signal;
receive an orientation signal;
form a plurality of filtered sensor signals by, at least in part, filtering the received sensor signal with a plurality of respective band-pass filters;
identify an activity class based, at least in part, on the plurality of filtered sensor signals;
determine a tilt indication based, at least in part on the received orientation signal and on the identified activity class; and
output one or more signals indicative of the identified activity class and the determined tilt indication.

27. The system of claim 26, wherein the received orientation signal comprises a quaternion.

28. The system of claim 26, wherein the at least one module is operable to determine a tilt indication by, at least in part:
determining a tilt change relative to a reference tilt; and
comparing the determined tilt change to a tilt threshold.
